(19) United States
(12) Patent Application Publication
(10) Pub. No.: US 2009/0047734 A1
(43) Pub. Date: Feb. 19, 2009

(54) METHOD OF SEPARATION OF DEOXYRIBONUCLEIC ACIDS

(75) Inventors: Mikael Berg, Uppsala (SE); Hans Blom, Uppsala (SE); Raf Lemmens, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE Healthcare Bio-Sciences AB, Uppsala (SE)

(21) Appl. No.: 12/282,668

(22) PCT Filed: Mar. 29, 2007
(86) PCT No.: PCT/SE07/00302

§ 371 Date: Sep. 12, 2008

(30) Foreign Application Priority Data
Mar. 31, 2006 (SE) 0600743-9

(51) Int. Cl. B01D 15/08; C07H 21/00; C12N 15/64

(52) U.S. Cl. 435/320.1; 536/25.4; 210/198.2

(57) ABSTRACT

The present invention relates to a method of separating nucleic acids from a liquid, which comprises providing a mobile phase comprising nucleic acids and a first salt, which forms lyotropic ions when dissolved; passing said mobile phase across a hydrophobic interaction chromatography (HIC) matrix to adsorb deoxyribonucleic acid(s); passing an eluent across said matrix to desorb one or more deoxyribonucleic acid(s), which eluent comprises the first salt as well as an increasing gradient provided by a second salt, which second salt forms less lyotropic ions when dissolved than the first salt; and isolating at least one fraction comprising separated deoxyribonucleic acid(s).

### TECHNICAL FIELD

The present invention relates to separation of nucleic acid molecules, especially plasmids, from other components in a solution. The method provides a novel scheme of desorbing the adsorbed molecules, which greatly improves the separation efficiency of the method all in all. The present method is preferably a chromatographic process.

### BACKGROUND

In the last decade, the administration of therapeutic genes to patients has become a reality for preventing or treating various diseases. Non-viral vectors are often preferred in clinical applications to minimise the risk of viral infections. This increases the demand for highly purified plasmids for use in gene therapy and plasmid-based vaccines, where the isoform of supercoiled DNA, also known as covalently closed circular (ccc) DNA, is predominately utilized. The stringent guidelines and rules set forth by health authorities require homogeneous preparations of purified supercoiled plasmid DNA for clinical applications.

Precipitation methods have been suggested for the purification of nucleic acids, such as plasmids.

A more commonly used purification method in the biotech field is chromatography. The term chromatography embraces a family of closely related separation methods, which are all based on the principle that two mutually immiscible phases are brought into contact. More specifically, the target compound is introduced into a mobile phase, which is contacted with a stationary phase. The target compound will then undergo a series of interactions between the stationary and mobile phases as it is being carried through the system by the mobile phase. The interactions exploit differences in the physical or chemical properties of the components in the sample.

Schluep et al suggested affinity chromatography, which is based on sequence-specific interactions between an immobilised synthetic oligonucleotide and a stretch of the plasmid DNA, for plasmid purification (see Schluep, T., and Cooney, C. L. Purification of plasmids by triplex affinity interaction. Nucleic Acids Research 1998; 26, 4524-4528). However, this technique cannot provide a homogeneous preparation of supercoiled plasmid DNA in sufficient quantities for non-analytical applications.

WO 02/083893 (Nyhammar et al) describes thiophilic chromatography for the binding of the different isoforms of plasmid DNA to a matrix. More specifically, chromatography ligands comprising sulphur as well as a pyridine, which ligands are sometimes denoted aromatic thioethers or S-aryl ligands, were utilized to isolate covalently closed circular (ccc) DNA. In fact, a separation matrix comprising these ligands was shown to efficiently separate covalently closed circular (ccc) plasmid DNA from its isoform, i.e. open circular (oc) form in a single chromatography step. Accordingly, the use of these matrices appears to be promising and should facilitate the production of highly purified supercoiled plasmid DNA for use in gene therapy and DNA vaccine applications.

U.S. Pat. No. 5,057,426 (Diagen) relates to the purification of nucleic acids using ion exchange. More specifically, a method is disclosed, wherein long-chain nucleic acids are separated from other substances in solutions containing nucleic acids and other materials by fixing long-chain nucleic acids in a nucleic acid-containing solution onto a porous matrix; washing the porous matrix to separate the other substances from the long-chain nucleic acids; and removing the fixed long-chain nucleic acids from the porous matrix. However, nucleic acids are generally known to bind relatively hard to ion exchange ligands, and it is consequently often difficult to elute the desired product.

U.S. Pat. No. 6,641,160 (Takashi et al) relates to separation of plasmids from contaminants using a tandem system comprising a first chromatography column, which is weakly hydrophobic, and a second chromatography column, which is strongly hydrophobic. The first column material adsorbs protein and RNA at a salt concentration at which plasmid is not adsorbed and produces an eluate containing plasmid and DNA. The second column material adsorbs plasmid and DNA from the eluate at a salt concentration at which the second adsorbs plasmid and DNA; hence the two columns can be attached serially. The second column is eluted, after disconnecting the first, by decreasing the salt concentration. Ion exchange chromatography is carried out in addition to the hydrophobic interaction chromatography by adsorbing plasmid from the eluate from the second column. Thus, the U.S. Pat. No. 6,641,160 patent utilises the classic HIC conditions of adsorption at high concentration of salt and desorption by decreasing the salt concentration.

US 20040038393 (Duarte et al) relates to the purification of double stranded plasmid DNA by binding impurities contained in clarified cell lysate to non-commercial hydrophobic interaction chromatography solid supports. More specifically, US 20040038393 relates to a process for the production of high purity plasmid DNA, wherein plasmid DNA is produced by cells; disrupted to obtain a lysate; concentrated by precipitation; purified using hydrophobic interaction chromatography (HIC); and finally concentrated by buffer exchange. In the purification step, plasmid DNA is not adsorbed, it can be collected at the outlet of the column.

Diogo et al (M. M. Diogo, J. A. Queiroz, and D. M. F. Prazeres in Journal of Chromatography A, 1069 (2005) 3-22: Chromatography of plasmid DNA) gives an overview of the use of plasmid DNA (pDNA) chromatography. Applications of the different types of chromatography to the purification of therapeutic pDNA are reviewed, and main advantages and disadvantages behind each technique are highlighted. More specifically, size-exclusion chromatography, slalom chromatography, anion exchange chromatography, hydroxy-apatite chromatography, reversed-phase chromatography, hydrophobic chromatography, and thiophilic adsorption chromatography are reviewed. With regard to hydrophobic interaction chromatography (HIC), experiments carried out on Sepharose™ CL-6B derivatised with a mildly hydrophobic ligand (1,4-butanediol diglycidyl ether) showed that in the presence of 1.5M of ammonium sulphate, total pDNA eluted in the void volume whereas impurities eluted later, well separated from the pDNA peak. The authors of the article explain this behaviour by the fact that pDNA molecules have the hydrophobic bases packed and shielded inside the double helix, and thus the interaction with the HIC media is minimal. When supercoiled (ccc) and open circular (oc) isoforms of pDNA were separated on a column comprising butyl ligands, high salt concentrations (3M ammonium sulphate) were required to bind the pDNA onto the resin surface and a reverse salt gradient was used to elute the isoforms sequentially. The article concludes that HIC may be used for pDNA or impurity capture; pDNA concentration; and quantitation, but that disadvantages are that in some cases, pDNA is eluted in high salt, which requires a subsequent desalting; and that in some cases, pDNA elutes in the flow through and is diluted.

Bratty et al (“Characterization of a chemically synthesized RNA having the sequence of the yeast initiator tRNAMet” FEBS 1990, vol. 269) relates to the chemical synthesis and subsequent purification of the Saccharomyces cerevisiae initiator tRNA, which is a long ribonucleotide. The purification involved chromatography on BND-cellulose, wherein binding was achieved at a lower conductivity and elution was achieved by increasing the conductivity. Bratty et al concludes that full chemical synthesis can be used to produce large, biologically active RNA species.

In conclusion, even though there are available methods for the purification of nucleic acids such as plasmid DNA, they tend to suffer from disadvantages such as low performance; cumbersome protocols including a large number of steps; lack of robustness etc. Thus, there is a clear need in this field of alternative methods that will suit the user in certain situations.

### SUMMARY OF THE PRESENT INVENTION

One aspect of the invention is to provide a novel method of separating nucleic acids from a solution using a hydrophobic interaction chromatography (HIC) matrix. This can be achieved by the method disclosed in the appended claims.

A specific aspect of the invention is to provide a method of desorbing nucleic acids from HIC matrices.

Another aspect of the invention is to provide a method for the separation of deoxyribonucleic acid(s), such as plasmid DNA, from one or more other components, such as proteins, RNA, and/or endotoxins.

A specific aspect of the invention is to provide a protocol for the isolation and/or purification of supercoiled plasmid DNA that reduces the tedious efforts for optimisation of the upstream processes. This may be achieved by the method described above, which is capable of separating the different plasmid isoforms from each other, such as supercoiled (also known as covalently closed circular) plasmid DNA from open circular plasmid DNA.

Further aspects and advantages of the present invention will appear from the specification and claims that follow below.

### Definitions

The term “nucleic acid molecules” is used herein synonymously with the term “nucleotides” and includes DNA, such as plasmid DNA and genomic DNA; RNA, such as mRNA, tRNA, sRNA and RNAi; and PNA.

The term “plasmid DNA” encompasses covalently closed circular (ccc) plasmid DNA, which is also known as supercoiled plasmid DNA, and open circular (oc) DNA. The term “nicked” plasmid DNA means herein the relaxed circle of DNA that is produced when one strand of the DNA being nicked, which relaxes the torsional strain needed to maintain supercoiling.

“Hydrophobic Interaction Chromatography” (HIC) is based on hydrophobic attraction between the stationary phase and the target. Thus, the term “hydrophobic interaction chromatography” refers to a method of separating target compounds or molecules based on the strength of their relative hydrophobic interactions with a hydrophobic separation matrix. In this context, “hydrophobicity” is defined as the repulsion between a non-polar compound and a polar environment.

The phrase to “pass a solution/eluent across a matrix” means any way of contacting a matrix with a solution or eluent followed by removal thereof. Accordingly, the term includes dynamic chromatographic methods as well as batch procedures.

The term “eluent” is used herein in its conventional meaning in chromatography, i.e. a solution capable of perturbing the interaction between the solid phase (separation matrix) and target (nucleic acids) and promoting selective dissociation of the target from the solid phase.

The term “lyotropic” is a measure of the ability of ions to influence the hydrophobic character of the interactions in a solvent.

The term “proteineous” as used herein includes whole protein as well as parts or traces of protein and other molecules comprising a peptidic structure.

### BRIEF DESCRIPTION OF THE DRAWINGS

FIG. 1 shows a chromatogram obtained after group separation of alkaline lysate, resulting in separate plasmid DNA and RNA fractions.

FIGS. 2a-b show the results of comparative examples, using a conventional HIC elution pattern. More specifically, FIG. 2 illustrate elution patterns in a water gradient of plasmid DNA adsorbed to Butyl Sepharose™ FF and Butyl-S Sepharose™ FF.

FIG. 3a-c show the results of a method according to the present invention. More specifically, FIG. 3 illustrates the elution pattern of plasmid DNA adsorbed to different hydrophobic Butyl Sepharose™ matrices by stepwise elution with increased ionic strength.

### DETAILED DESCRIPTION OF THE PRESENT INVENTION

A first aspect of the present invention relates to the separation of nucleic acids, such as isolation or purification of nucleic acids, advantageously deoxyribonucleic acid(s), from other components of a liquid, such as a lysate. More specifically, the invention relates to a method of separating one or more nucleic acids from other components of a liquid, which method comprises the use of a hydrophobic interaction chromatography (HIC) matrix, wherein the nucleic acid(s) are eluted by an increasing salt gradient. Thus, the nucleic acids are adsorbed to the matrix via hydrophobic interactions between nucleic acid and matrix.

The first aspect also encompasses a method of separating one or more nucleic acids from other components of a liquid, which method comprises adsorbing the nucleic acid(s) at a first salt concentration and desorbing the nucleic acid(s) at a second salt concentration which is higher than the first salt concentration. In the preferred embodiment, the invention relates to a method of separating one or more nucleic acids from a liquid, which method comprises using a hydrophobic interaction chromatography (HIC) matrix and eluting the nucleic acid(s) by an increasing salt gradient, wherein the nucleic acid is deoxyribonucleic acid(s) (DNA).

In one embodiment, the method according to the invention comprises
(a) providing a mobile phase comprising at least one deoxyribonucleic acid (DNA) and a first salt, which forms lyotropic ions when dissolved;
(b) passing said mobile phase across a hydrophobic interaction chromatography (HIC) matrix to adsorb one or more deoxyribonucleic acid(s) to the matrix;
(c) optionally, washing the matrix;
(d) passing an eluent across said matrix to desorb one or more deoxyribonucleic acid(s), which eluent in addition to the first salt also comprises a gradient of increasing ionic strength resulting from an increasing concentration of a second salt, which forms less lyotropic ions when dissolved than the first salt; and
(e) isolating at least one fraction comprising separated deoxyribonucleic acid(s).

In an advantageous embodiment, the deoxyribonucleic acid(s) are separated from other components of the cells from which they originate. The cells may be prokaryotic or eukaryotic, such as recombinant cells designed to produce a specified nucleic acid. In a specific embodiment, the present method comprises a first step of disintegrating the cells to provide a liquid comprising nucleic acids. Such disintegration is advantageously performed by chemical or mechanical lysis, which are both well known methods in this field. The lysed cells may be filtered or centrifuged before the chromatographic process according to the invention. Accordingly, in one embodiment, the mobile phase is preferably essentially free of cell debris.

In one embodiment of the present method, the deoxyribonucleic acid fraction isolated according to the invention comprises plasmid DNA. In an advantageous embodiment, said fraction is comprised of substantially pure plasmid DNA.

In another embodiment, the deoxyribonucleic acid fraction isolated according to the invention comprises one single plasmid DNA isoform. Thus, in one embodiment, the isolated deoxyribonucleic acid fraction comprises supercoiled plasmid DNA essentially free of open circular plasmid DNA.

In yet another embodiment, the deoxyribonucleic acid fraction isolated is essentially free of at least one species selected from the group comprising of proteineous components; RNA; and endotoxins. In an advantageous embodiment, the plasmid DNA, such as supercoiled plasmid DNA, is essentially free from RNA.

The present inventors have found that RNA and endotoxins which are present as impurities in the mobile phase can easily be eluted simply by adding a liquid, known as the eluent, which decreases the salt concentration, as conventionally carried out to elute the desired sample from a HIC matrix Thus, the present invention also encompasses an embodiment, which involves adding an aqueous eluent without any salts, or a low salt buffer, to elute impurities before eluting the target deoxyribonucleic acid(s). Such elution may also be used e.g. for regeneration of the separation matrix, i.e. after elution of target deoxyribonucleic acid(s). However, in an alternative embodiment, the nucleic acid(s) separated according to the present is RNA, which may be used e.g. for scientific research. In this embodiment, deoxyribonucleic acid (DNA) is regarded the impurity together with proteineous components, endotoxins etc.

Thus, the present invention shows that it is possible to separate nucleic acid such as DNA and/or RNA from other components of a liquid such as proteineous components and endotoxin using a novel elution principle to release adsorbed components from hydrophobic interaction chromatography (HIC) matrices. The method according to the invention is useful for purification purposes, such as the purification of plasmid DNA for use in gene therapy and nucleic acids for use as DNA vaccines. The purification according to the invention may be laboratory scale or large scale for bioprocess purposes. The purified nucleic acid will be substantially free from other components, such as 95% pure, preferably 98% pure, more preferably about 99% pure and most preferably about 100% pure. Thus, in an advantageous embodiment, the present method will provide isolated supercoiled plasmid DNA of acceptable gene therapy grade.

Alternatively, or in addition, the present invention is used for analytical purposes, such as to analyse nucleotide mixtures in solution. In this embodiment, the term “separation” refers to the detection and/or quantitation. In an advantageous embodiment, the method is used in the diagnostic industry such as in a method wherein a certain disease or condition is diagnosed by separating a nucleic acid and subsequent quantitation and/or analysis thereof.

In an advantageous embodiment of the present method, the lyotropic and less lyotropic ions referred to in steps (a) and (d) are the anions formed by dissolution of the first and second salts, respectively.

In one embodiment of the present method, the mobile phase provided in step (a) comprises a dissolved alkali salt, such as ammonium sulphate or sodium sulphate. Accordingly, the anions present therein during the adsorption will be sulphate ions, which are strongly lyotropic. In a specific embodiment, the concentration of said salt is below about 3.0 M, such as about 2.5 M. As the skilled person in this field will realise, the upper limit is determined by the solubility of each salt. However, as the skilled in this field will realise, different salt concentrations may be required in different situations, not only depending on the specific ligand used on the matrix, but also on the nucleic acid to be separated. The pH of the mobile phase during adsorption step may differ from the pH of the eluent. However, it is understood that to ensure the stability of the nucleic acids to be separated, the pH should remain at physiological pH, i.e. in the range of about 6.5-8.5.

In an advantageous embodiment, the HIC matrix to which the desired nucleic acids have been adsorbed is washed before elution according to step (d). During washing, loosely bound contaminants are removed. Such washing can easily be performed by the skilled in this field according to standard procedures.

Step (d) according to the present invention is performed with a suitable eluent, such as an aqueous eluent, which in addition to the first salt that forms lyotropic ions also comprises a second salt, whereby the salt concentration is increased to desorb the nucleic acids from the HIC matrix. As is well known, the increased salt concentration will result in increased conductivity. Thus, in one embodiment, the eluent comprises a gradient of increasing ionic strength, such as a continuous or step-wise gradient. As is well known, gradients of the salt are formed by mixing two buffers, one containing a high concentration of the salt and one containing a low concentration of this salt. But for their salt contents, the two eluents are identical.

The increasing ionic strength of the eluent used in step (d) is provided by an increasing concentration of the second salt in the eluent, which second salt is capable of forming less lyotropic ions, such as anions, than the first salt. As is well known to the skilled in this field, the lyotropic properties of ions can be rated according to the Hofmeister or lyotropic series. The dissolved second salt will gradually change the conditions in the solvent, which change was found by the present inventor to be useful to separate DNA from RNA and/or endotoxins; and plasmid DNA isoforms from each other. In fact it was found that for most ligands, RNA is eluted as the last fraction simply by water. Proteins and endotoxins can only be removed by the reduction of ionic strength, indicating a solely hydrophobic binding mechanism of these molecules to the matrix, completely different from the mechanisms nucleic acids deploy. In one embodiment, the second salt dissolved in the eluent is an alkali salt, such as sodium or potassium chloride. Thus, in this embodiment, the less lyotropic anions are chloride ions. In a specific embodiment, the maximum concentration of the salt is about 3 M. However, as mentioned above in relation to the salt dissolved in the adsorption solution, for each specific ligand and desired nucleic acid, some routine testing may be needed in order to determine optimal conditions. As mentioned above, the pH value during elution should be maintained within a range where the nucleic acids are stable, e.g. to avoid nicked forms of plasmid DNA.

As appears from the above, the present invention involves applying novel elution conditions to a hydrophobic interaction chromatography (HIC) matrix. As is well known, conventional hydrophobic interaction chromatography (HIC) is a technique for the purification and separation of biomolecules based on differences in their surface hydrophobicity. However, in conventional HIC, the bound target is released from the matrix by decreasing the concentration of lyotropic salt. Contrary, the present invention shows that in the separation of nucleic acids, such as plasmid DNA, an efficient elution is obtained by instead increasing the salt concentration to elute adsorbed nucleic acids.

The HIC matrix used in the present method can be of any conventionally used kind. The carrier may be made of any material conventionally used for HIC matrices, such as a weakly or strongly hydrophobic material. Thus, in one embodiment, the carrier is substantially hydrophobic. In a specific embodiment, the carrier comprises styrene and divinylbenzene (DVB). Conventionally used hydrophobic carriers involve synthetic polymeric materials such as cross-linked polymeric materials. In one embodiment, the base matrix is comprised of cross-linked synthetic polymers, such as styrene or styrene derivatives, divinylbenzene, acrylamides, acrylate esters, methacrylate esters, vinyl esters, vinyl amides etc. Such polymeric carriers are easily produced according to standard methods, see e.g. “Styrene based polymer supports developed by suspension polymerization” (R Arshady: Chimica e L'Industria 70(9), 70-75 (1988)). Commercially available hydrophobic carriers are equally useful, such as the SOURCE™ series available from GE Healthcare, Uppsala, Sweden.

Alternatively, the HIC matrix used in the present method is a hydrophilic carrier coated with hydrophobic ligands. The hydrophilic carrier may be comprised of a cross-linked carbohydrate material, such as agarose, agar, cellulose, dextran, chitosan, konjac, carrageenan, gellan, alginate etc. Such carriers are easily prepared by the skilled person according to standard methods, such as inverse suspension gelation (S Hjerten: Biochim Biophys Acta 79(2), 393-398 (1964). Alternatively, the carrier is a commercially available carrier, such as Sepharose™ FF (GE Healthcare, Uppsala, Sweden). Hydrophobic ligands are easily coupled to such hydrophilic carriers using well known methods in this field, see e.g. Immobilized Affinity Ligand Techniques, Hermanson et al, Greg T. Hermanson, A. Krishna Mallia and Paul K. Smith, Academic Press, INC, 1992. In one embodiment, the HIC matrix comprises hydrophobic ligands coupled to a carrier.

The hydrophobic ligands may be any commonly used ligands, such as linear or branched carbon chains, which may be substituted or non-substituted. Alternatively, the ligands comprise aromatic groups, such as cyclic aromatic groups. Thus, in one embodiment, the ligands comprise alkyl groups, such as butyl or octyl groups. In another embodiment, the ligands comprise aromatic groups, such as phenyl groups. In a specific embodiment, the HIC matrix used in the present method comprises multimodal chromatography ligands, which comprise at least one hydrophobic group and one or more additional group that adds one or more further functionalities such as a hydrogen-bonding group and/or an ion exchanger, preferably an anion exchanger.

Alternatively, the present method uses a commercially available HIC matrix, such as Butyl Sepharose™; Butyl-S Sepharose™; TSKgel Butyl-NPR or the like.

The carrier may be of any format conventionally used in chromatography, such as porous or non-porous particles, such as irregular or essentially spherical particles; monoliths; membranes, such as stacked membranes; filters; surfaces; capillaries; microtiter plates etc.

Thus, in one embodiment, the separation matrix is comprised of essentially spherical particles. The present method may then be performed with the matrix arranged in the form of an expanded bed or a packed bed, and can be dynamic, i.e. chromatography, or run in a batch mode. In packed bed adsorption, the matrix is packed in a chromatographic column and all solutions used during a purification process are passed through the column, usually in the same direction. In expanded bed adsorption however, the matrix is expanded and equilibrated by applying a liquid flow through the column, usually from beneath. A stable fluidised expanded bed is formed when there is a balance between particle sedimentation or rising velocity and the flow velocity during application of the sample and washing steps. In the elution step of an expanded bed, the matrix is precipitated and behaves like a packed bed matrix.

In one embodiment, the matrix particles are of a mean size in the range of about 10-300 μm, e.g. within a range of 10-20, 20-50, 50-100, 100-200 or 200-300 μm. However, the particles can advantageously be prepared in any size for which commercially available sieve equipment is available, such as 250, 212, 180, 150, 125, 106, 90, 75, 63, 45, 37, 30, 25, 20, 15 μm.

In an alternative embodiment, the separation matrix is a membrane. Such a membrane may be used to adsorb nucleic acids which are not the desired product but a contaminant in a specific process, in which case the membrane is preferably a disposable which is discarded after use, eliminating more contact with the removed contaminant.

### DETAILED DESCRIPTION OF THE DRAWINGS

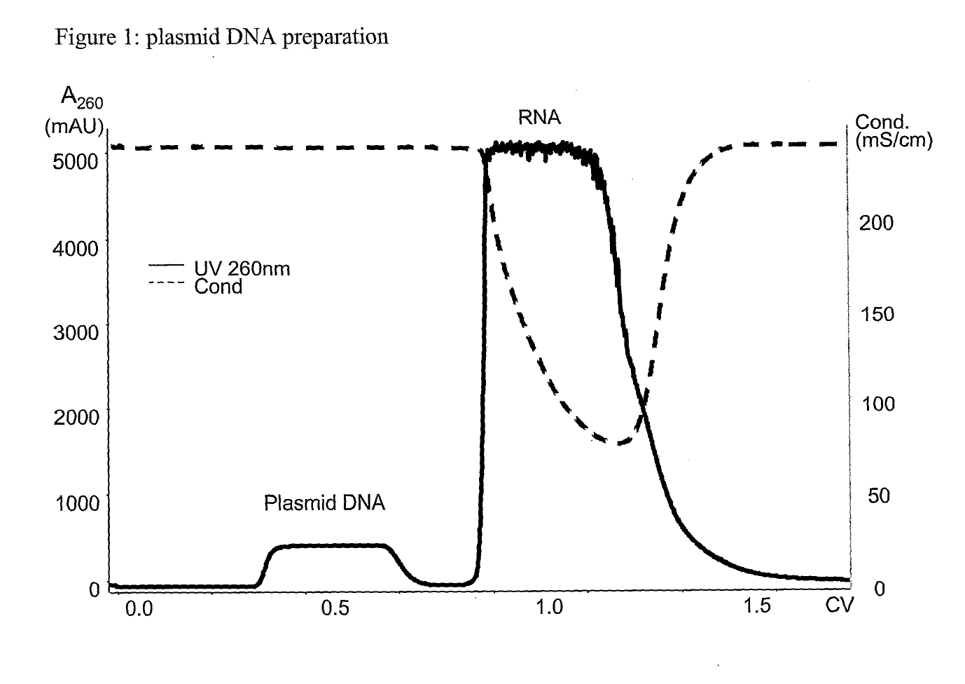

FIG. 1 shows a chromatogram obtained after group separation of alkaline lysate, resulting in separate plasmid DNA and RNA fractions, as described in Example 1 below.

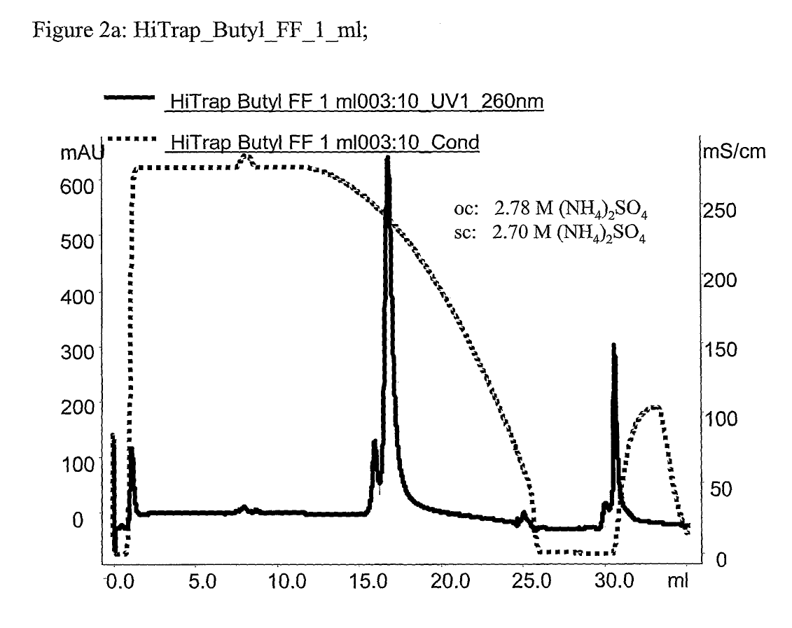

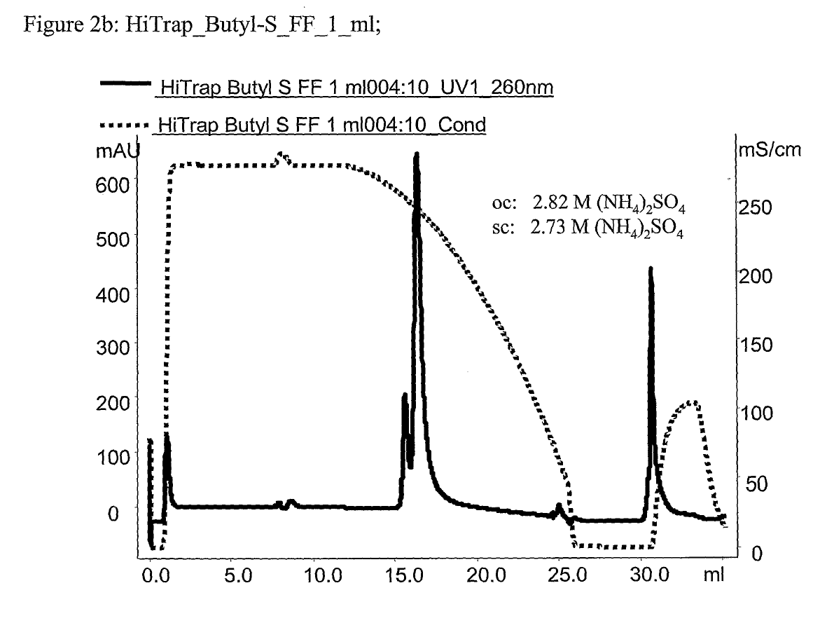

FIGS. 2a-b illustrates how conventional elution was used to determine the lowest common conductivity for adsorption to the tested HIC matrices, as described in Example 2 below. The eluent was a water gradient, which desorbed the plasmid DNA which had earlier been adsorbed to Butyl Sepharose™ FF and Butyl-S Sepharose FF™. More specifically, FIGS. 2a-b shows chromatograms where pJV4 plasmid in 3.0 M (NH₄)₂SO₄, 10 mM EDTA, 100 mM Tris-HCl, pH 7.0 was loaded on the columns: FIG. 2a) shows results from using a 1 ml HiTrap column prepacked with Butyl Sepharose™ FF; FIG. 2b) shows results from using a 1 ml HiTrap column prepacked with Butyl-S Sepharose™ FF.

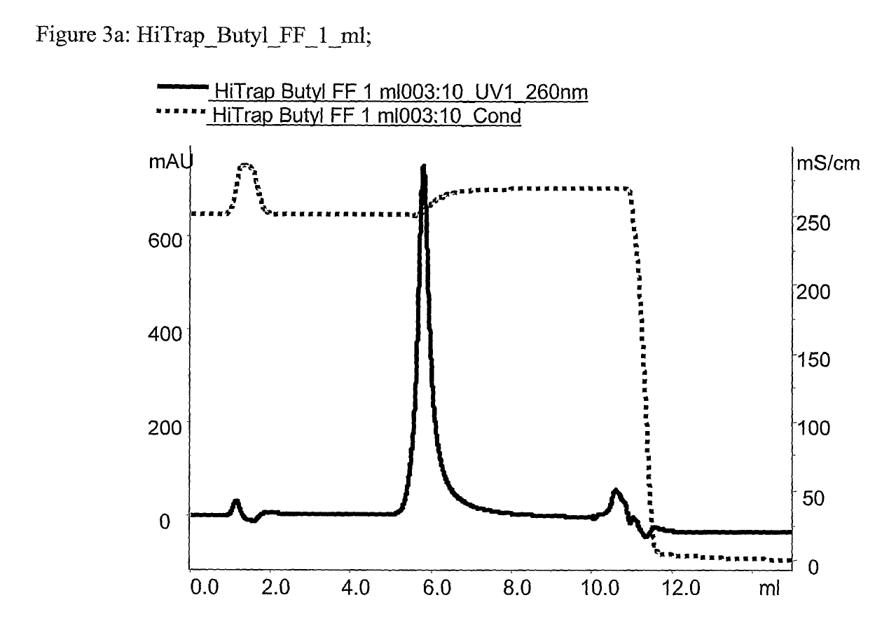

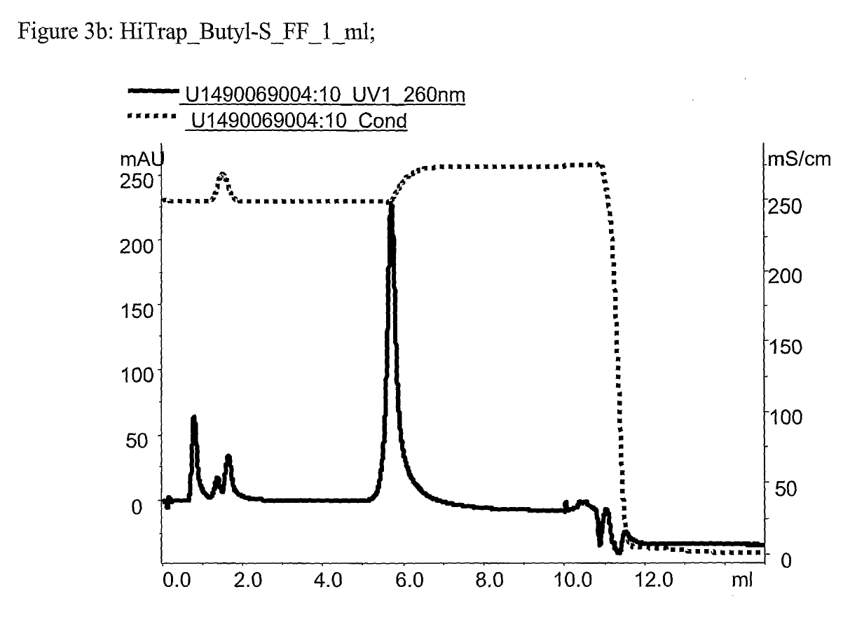

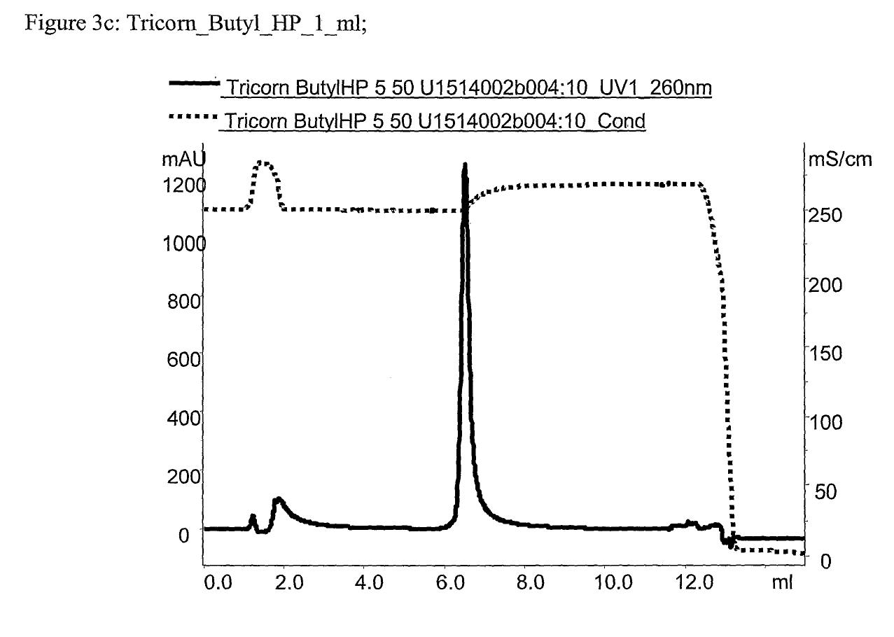

FIGS. 3a-c illustrates the elution pattern of plasmid DNA adsorbed to different Butyl Sepharose™ matrices by stepwise elution with increased ionic strength, in accordance with the present invention and as described in Example 3 below. More specifically, a mobile phase comprising plasmid DNA in 3.0 M (NH₄)₂SO₄, 10 mM EDTA, 100 mM Tris-HCl, pH 7.0 was applied to columns prepacked with three different HIC matrices: FIG. 3a) shows results from a HiTrap column prepacked with Butyl Sepharose™ FF media; FIG. 3b) shows results from a HiTrap column prepacked with Butyl-S Sepharose™ FF media; and FIG. 3c shows results from a Tricorn™ column prepacked with Butyl Sepharose™ HP media, all preconditioned in 2.25 M (NH₄)₂SO₄, 10 mM EDTA, 100 mM Tris-HCl, pH 7.0. As appears from FIG. 3, all the tested matrices show similar elution profiles. Thus, increasing the conductivity by increasing the salt concentration in the elution buffer enables desorption of plasmid DNA from hydrophobic interaction chromatography (HIC) matrices. Without wishing to be bound to any specific theory, this concept is contrary to the accepted behaviour of biological molecules such as proteins in interaction to hydrophobic matrices.

### Experimental Part

Below the present invention will be disclosed by way of examples, which are intended solely for illustrative purposes and should not be construed as limiting the present invention as defined in the appended claims. All references mentioned below or elsewhere in the present application are hereby included by reference.

### EXAMPLE 1

### Preparation of Plasmid DNA

A 6 k base pair plasmid pJV4 plasmid was transfected and grown in E. coli by well established protocols (Sambrook, J., and Russel, D. W. Molecular cloning: a laboratory manual, Cold Spring Harbor, N.Y., 2001). Clarified alkaline lysate was prepared according to Horn, N. A., Meek, J. A., Budahazi, G., and Marquet, M. Cancer gene therapy using plasmid DNA: purification of DNA for human clinical trials. Human Gene Therapy 1995; 6, 565-573). A crude alkaline plasmid DNA preparation (containing both open circular and supercoiled isoforms) was prepared and loaded on a Sepharose™ 6 Fast Flow column preconditioned in to 2.0 M (NH₄)₂SO₄, 10 mM EDTA, 100 mM Tris-HCl, pH 7.0. After chromatography at a flow rate of 50 cm/h, plasmid DNA was separated from RNA.

### EXAMPLE 2

### Chromatography Using Conventional HIC Elution

The concentration of ammonium sulphate in the above-described plasmid DNA sample was increased to 3.0 M (NH₄)₂SO₄ by addition of solid salt to provide conditions for full binding to the hydrophobic interaction chromatography (HIC) matrices used. The columns below are obtainable prepacked with HIC matrix (media) from GE Healthcare, Uppsala, Sweden. Each tested column was preconditioned in 3.0 M (NH₄)₂SO₄, 10 mM EDTA, 100 mM Tris-HCl, pH 7.0. FIGS. 2a-b shows chromatograms wherein pJV4 plasmid in 3.0 M (NH₄)₂SO₄, 10 mM EDTA, 100 mM Tris-HCl, pH 7.0 was loaded on the columns as follows: FIG. 2a) 1 ml HiTrap column packed with Butyl Sepharose™ FF; and FIG. 2b) 1 ml HiTrap column packed with Butyl-S Sepharose™ FF. The lowest common conductivity for binding to all of the tested columns was determined by elution with a water gradient. The range for elution start of open circular plasmid DNA was determined to the range of 2.83 to 2.67 M (NH₄)₂SO₄ while the range for elution start of supercoiled plasmid DNA was determined to the range of 2.73 to 2.52 M (NH₄)₂SO₄.

### EXAMPLE 3

### Chromatography According to the Invention

Mobile phase comprising the above-described plasmid DNA in 3.0 M (NH₄)₂SO₄, 10 mM EDTA, 100 mM Tris-HCl, pH 7.0 was applied to columns packed with a three different Butyl Sepharose™ matrices as follows: FIG. 3a) shows the results from a HiTrap column packed with Butyl Sepharose™ FF; FIG. 3b) shows results from a HiTrap column packed with Butyl-S Sepharose™ FF; and FIG. 3c) shows results from a Tricom column packed with Butyl Sepharose™ HP, preconditioned in 2.25 M (NH₄)₂SO₄, 10 mM EDTA, 100 mM Tris-HCl, pH 7.0. After washing with preconditioning buffer, the elution according to the invention was performed by increasing the conductivity with 2.0 M NaCl, 2.25 M (NH₄)₂SO₄, 10 mM EDTA, 100 mM Tris-HCl, pH 7.0. As appears from FIG. 3, plasmid DNA was sharply eluted from all the tested columns upon increasing the conductivity.

1. A method of separating one or more nucleic acids from a liquid, which method comprises using a hydrophobic interaction chromatography (HIC) matrix and eluting the nucleic acid(s) by an increasing salt gradient, wherein the nucleic acid is deoxyribonucleic acid(s).

2. The method of claim 1, comprising adsorbing the deoxyribonucleic acid(s) at a first salt concentration; and desorbing the deoxyribonucleic acid(s) at a second salt concentration which is higher than the first salt concentration.

3. The method of claim 1, comprising providing a mobile phase comprising at least one deoxyribonucleic acid(s) and a first salt, which first salt forms lyotropic ions when dissolved;
(a) passing said mobile phase across a hydrophobic interaction chromatography (HIC) matrix to adsorb one or more deoxyribonucleic acid(s);
(b) optionally washing the matrix;
(c) passing an eluent across said matrix to desorb one or more deoxyribonucleic acid(s), which eluent in addition to the first salt also comprises a gradient of increasing ionic strength provided by an increasing concentration of a second salt, which second salt forms less lyotropic ions when dissolved than the first salt; and
(d) isolating at least one fraction comprising separated deoxyribonucleic acid(s).

4. The method of claim 3, wherein the isolated deoxyribonucleic acid(s) fraction comprises plasmid DNA.

5. The method of claim 4, wherein the isolated deoxyribonucleic acid(s) fraction comprises covalently closed circular (supercoiled) plasmid DNA essentially pure from other plasmid isoforms such as open circular plasmid DNA.

6. The method of claim 3, wherein in the isolated fraction, the deoxyribonucleic acid(s) is essentially free from proteineous components and/or RNA and/or endotoxins.

7. The method of claim 3, wherein the lyotropic and less lyotropic ions referred to in step (a) and (d) are the anions formed by dissolution of the first and second salts, respectively.

8. The method of claim 3, wherein the first salt is ammonium sulphate or sodium sulphate.

9. The method of claim 3, wherein the concentration of the first salt in the mobile phase is below about 3 M.

10. The method of claim 3, wherein the second salt is an alkali salt, such as sodium chloride or potassium chloride.

11. The method of claim 3, wherein the maximum concentration of the second salt in the eluent is about 3 M.

12. The method of claim 1, wherein the HIC matrix comprises hydrophobic ligands coupled to a carrier.

13. The method of claim 12, wherein the ligands comprises alkyl groups.

14. The method of claim 12, wherein the carrier is substantially hydrophobic.

15. The method of claim 14, wherein the carrier comprises styrene and divinylbenzene (DVB).

16. A kit for plasmid DNA purification, which comprises, in separate compartments, a chromatography column comprising a HIC matrix; an adsorption buffer; an eluent having a higher salt concentration than the adsorption buffer; and written instructions.
